(19) United States
(12) Patent Application Publication
(10) Pub. No.: US 2026/0041822 A1
(43) Pub. Date: Feb. 12, 2026

(54) Bottlebrush and Multi-Arm Polymers for Medical Applications

(71) Applicant: Boston Scientific Scimed, Inc., Maple Grove, MN (US)

(72) Inventors: Nicolas BALL-JONES, Minneapolis, MN (US); Sasha Viola RIOS, Minneapolis, MN (US); Yen-Hao HSU, Shrewsbury, MA (US); Joseph Thomas DELANEY, JR., Minneapolis, MN (US)

(73) Assignee: Boston Scientific Scimed, Inc., Maple Grove, MN (US)

(21) Appl. No.: 18/747,038

(22) Filed: Jun. 18, 2024

Related U.S. Application Data
(60) Provisional application No. 63/509,483, filed on Jun. 21, 2023.

(51) Int. Cl. A61L 31/06; A61L 31/14; C08J 3/24

(52) U.S. Cl. CPC A61L 31/06; A61L 31/145; C08J 3/24

(57) ABSTRACT

A reactive bottlebrush polymer for medical applications is described that comprises a polymer backbone and a plurality of polymer side chains linked to the polymer backbone, at least a portion of the side chains each comprising a hydrophilic polymer segment covalently linked to the polymer backbone and a reactive moiety covalently linked to the hydrophilic polymer segment at an end of the side chain opposite the polymer backbone. Also described for medical applications is a reactive multi-arm polymer that comprises three or more polymer arms that each comprise a hydrophilic polymer segment containing one or more types of polar aprotic vinyl monomer residues and a reactive moiety covalently linked to the hydrophilic polymer segment. A reactive polysaccharide for medical applications is further described that comprises a polysaccharide backbone that comprises free carboxyl groups and cyclic imide ester groups covalently attached along a length of the polysaccharide backbone.

### CROSS-REFERENCE TO RELATED APPLICATIONS

This application claims the benefit of U.S. Provisional Patent Application Ser. No. 63/509,483 filed on Jun. 21, 2023, the disclosure of which is incorporated herein by reference.

### FIELD

The present disclosure relates to reactive multi-arm polymers, reactive bottlebrush polymers and reactive polysaccharide polymers, to crosslinked hydrogels formed from such polymers, and to crosslinkable systems for forming such hydrogels, among other aspects. The reactive polymers, hydrogels and crosslinkable systems for forming the same are useful, for example, in various medical applications.

### BACKGROUND

Bioresorbable hydrogels with rapid crosslinking reaction rate in vivo, known by the trade name of SpaceOAR®, have become a prominent biomaterial and obtained clinical success in creating the space between prostate and rectum, tremendously improving patient safety during the cancer therapies. A further improvement based on this application is that some of 8-Arm PEG branches are functionalized with 2,3,5-triiiodobenzamide (TIB) groups, replacing part of the activated ester end groups, succinimidyl glutarate (SG), in order to provide intrinsic radiopacity to the hydrogels themselves for CT-visibility. This hydrogel, known by the trade name of SpaceOAR Vue®, is the next generation of SpaceOAR® for prostate medical applications.

### SUMMARY

The present disclosure provides an alternative approach to that described above.

In some aspects, the present disclosure provides a reactive bottlebrush polymer comprising a polymer backbone and a plurality of polymer side chains linked to the polymer backbone, at least a portion of the side chains each comprising a hydrophilic polymer segment covalently linked to the polymer backbone and a reactive moiety covalently linked to the hydrophilic polymer segment at an end of the side chain opposite the polymer backbone.

In some embodiments, which are applicable to the above aspects, the reactive moiety comprises an electrophilic group or alkenyl-containing group.

In some embodiments, which are applicable to the above aspects, the reactive moiety comprises a cyclic imide ester group, an imidazole ester group, an imidazole carboxylate group, a benzotriazole ester group, an acryloyl group, or a methacryloyl group.

In some embodiments, which are applicable to the above aspects and embodiments, the hydrophilic polymer segment is selected from a polyethylene-oxide-containing segment, poly(amino acid)-containing segment, a polyoxazoline-containing segment, or a hydrophilic polymer segment containing one or more types of polar aprotic vinyl monomer residues.

In some embodiments, which are applicable to the above aspects and embodiments, the hydrophilic polymer segment is an iodine-containing hydrophilic polymer segment.

In some embodiments, which are applicable to the above aspects and embodiments, the reactive bottlebrush polymer further comprises a cyclic anhydride residue disposed between the hydrophilic polymer segment and the reactive moiety. In some of these embodiments, the cyclic anhydride residue is an iodine-containing cyclic anhydride residue and/or the cyclic anhydride residue is selected from a residue of an iodine- or non-iodine-containing glutaric anhydride compound, a residue of an iodine- or non-iodine-containing succinic anhydride compound, a residue of an iodine- or non-iodine-containing malonic anhydride compound, a residue of an iodine- or non-iodine-containing adipic anhydride compound, and a residue of an iodine- or non-iodine-containing diglycolic anhydride compound.

In some embodiments, the present disclosure provides a crosslinked hydrogel composition that comprises a crosslinked reaction of (a) polyamine compound and (b) a reactive bottlebrush polymer in accordance with any of the above aspects and embodiments.

In some embodiments, the present disclosure provides a system for forming a hydrogel composition that comprises (a) polyamine compound and (b) a reactive bottlebrush polymer in accordance with any of the above aspects and embodiments. In some embodiments, the system comprises a first composition that comprises the polyamine compound, a second composition that comprises the reactive bottlebrush polymer and, optionally an accelerant composition.

In some embodiments, the present disclosure provides method of treatment comprising administering to a subject a mixture that comprises (a) a polyamine compound and (b) a reactive bottlebrush polymer in accordance with any of the above aspects and embodiments, under conditions such that the polyamine compound and the reactive bottlebrush polymer crosslink after administration.

In some embodiments, the present disclosure provides method of treatment comprising administering a reactive bottlebrush polymer in accordance with any of the above aspects and embodiments to a body of a subject under conditions such that the reactive bottlebrush polymer reacts with amines found naturally in or on the body of the subject after administration.

In other aspects, the present disclosure pertains to a reactive multi-arm polymer that comprises three or more polymer arms that each comprise a hydrophilic polymer segment containing one or more types of polar aprotic vinyl monomer residues and a reactive moiety covalently linked to the hydrophilic polymer segment.

In some embodiments, which are applicable to the above aspects, the reactive moiety comprises an electrophilic group or alkenyl-containing group.

In some embodiments, which are applicable to the above aspects, the reactive moiety comprises a cyclic imide ester group, an imidazole ester group, an imidazole carboxylate group, a benzotriazole ester group, an acryloyl group, or a methacryloyl group.

In some embodiments, which are applicable to the above aspects and embodiments, the hydrophilic polymer segment is selected from a polyethylene-oxide-containing segment, poly(amino acid)-containing segment, a polyoxazoline-containing segment, or a hydrophilic polymer segment containing one or more types of polar aprotic vinyl monomer residues.

In some embodiments, which are applicable to the above aspects and embodiments, the hydrophilic polymer segment is an iodine-containing hydrophilic polymer segment.

In some embodiments, which are applicable to the above aspects and embodiments, the reactive multi-arm polymer further comprises a cyclic anhydride residue disposed between the hydrophilic polymer segment and the reactive moiety. In some of these embodiments, the cyclic anhydride residue is an iodine-containing cyclic anhydride residue and/or the cyclic anhydride residue is selected from a residue of an iodine- or non-iodine-containing glutaric anhydride compound, a residue of an iodine- or non-iodine-containing succinic anhydride compound, a residue of an iodine- or non-iodine-containing malonic anhydride compound, a residue of an iodine- or non-iodine-containing adipic anhydride compound, and a residue of an iodine- or non-iodine-containing diglycolic anhydride compound.

In some embodiments, the present disclosure provides a crosslinked hydrogel composition that comprises a crosslinked reaction of (a) polyamine compound and (b) a reactive multi-arm polymer in accordance with any of the above aspects and embodiments.

In some embodiments, the present disclosure provides a system for forming a hydrogel composition that comprises (a) polyamine compound and (b) a reactive multi-arm polymer in accordance with any of the above aspects and embodiments. In some of these embodiments, embodiments, the system comprises a first composition that comprises the polyamine compound, a second composition that comprises the reactive multi-arm polymer and, optionally an accelerant composition.

In some embodiments, the present disclosure provides method of treatment comprising administering to a subject a mixture that comprises (a) a polyamine compound and (b) a reactive multi-arm polymer in accordance with any of the above aspects and embodiments, under conditions such that the polyamine compound and the reactive multi-arm polymer crosslink after administration.

In some embodiments, the present disclosure provides method of treatment comprising administering a reactive multi-arm polymer in accordance with any of the above aspects and embodiments to a body of a subject under conditions such that the reactive multi-arm polymer reacts with amines found naturally in or on the body of the subject after administration.

In other aspects, the present disclosure pertains to a reactive polysaccharide comprising a polysaccharide backbone that comprises free carboxyl groups and cyclic imide ester groups covalently attached along a length of the polysaccharide backbone.

In some embodiments, the cyclic imide ester groups are selected from succinimide ester groups, maleimide ester groups, glutarimide ester groups, phthalimide ester groups, and bicyclo[2.2.1]hept-5-ene-2,3-dicarboxylic acid imide ester groups.

In some embodiments, which are applicable to the above aspects and embodiments, the polysaccharide backbone is formed from a polysaccharide that contains one or more uronic acid species.

In some embodiments, which are applicable to the above aspects and embodiments, the polysaccharide backbone is formed from hyaluronic acid.

In some embodiments, the present disclosure provides a crosslinked hydrogel composition that comprises a crosslinked reaction of (a) polyamine compound and (b) a reactive polysaccharide in accordance with any of the above aspects and embodiments.

In some embodiments, the present disclosure provides a system for forming a hydrogel composition that comprises (a) polyamine compound and (b) a reactive polysaccharide in accordance with any of the above aspects and embodiments. In some of these embodiments, embodiments, the system comprises a first composition that comprises the polyamine compound, a second composition that comprises the reactive polysaccharide and, optionally an accelerant composition.

In some embodiments, the present disclosure provides method of treatment comprising administering to a subject a mixture that comprises (a) a polyamine compound and (b) a reactive polysaccharide in accordance with any of the above aspects and embodiments, under conditions such that the polyamine compound and the reactive polysaccharide crosslink after administration.

In some embodiments, the present disclosure provides method of treatment comprising administering a reactive polysaccharide in accordance with any of the above aspects and embodiments to a body of a subject under conditions such that the reactive polysaccharide reacts with amines found naturally in or on the body of the subject after administration.

Potential benefits associated with the present disclosure include one or more of the following: crosslink density is enhanced, in vivo persistence is obtained, and in some cases radiocontrast is maintained or enhanced.

The above and other aspects, embodiments, features and benefits of the present disclosure will be readily apparent from the following detailed description.

### BRIEF DESCRIPTION OF THE DRAWINGS

FIG. 1 schematically illustrates a method of forming a reactive poly(ethylene oxide) bottlebrush polymer, in accordance with an embodiment of the present disclosure.

FIG. 2 schematically illustrates a method of forming a reactive poly(amino acid) bottlebrush polymer, in accordance with an embodiment of the present disclosure.

FIG. 3 schematically illustrates a method of forming a reactive polyoxazoline bottlebrush polymer, in accordance with an embodiment of the present disclosure.

FIG. 4 schematically illustrates a method of forming a reactive hyaluronic acid polymer, in accordance with an embodiment of the present disclosure.

FIG. 5 schematically illustrates a method of forming a multi-functional RAFT agent, in accordance with an embodiment of the present disclosure.

FIG. 6 schematically illustrates a method of forming a multi-functional RAFT agent, in accordance with another embodiment of the present disclosure.

FIG. 7 schematically illustrates processes of aminolysis, thiol-Michael addition, and succinimidyl glutarate end capping, for use in various embodiments of the present disclosure.

FIG. 8 illustrates a delivery device, in accordance with an embodiment of the present disclosure.

FIG. 9 illustrates a delivery device, in accordance with another embodiment of the present disclosure.

### DETAILED DESCRIPTION

In various aspects, the present disclosure pertains to reactive polymers including reactive bottlebrush polymers and reactive multi-arm polymer.

Reactive bottlebrush polymers in accordance with the present disclosure include polymers that comprise a plurality of polymer side chains linked to a polymer backbone, at least a portion of the side chains having a reactive moiety covalently linked to an end of the side chain opposite the polymer backbone. In some embodiments, at least a portion of the polymer side chains comprise a hydrophilic polymer segment covalently linked to the linear polymer backbone, a cyclic anhydride residue covalently linked to the hydrophilic polymer segment, and a reactive moiety that is covalently linked to the cyclic anhydride residue.

In particular embodiments, the reactive bottlebrush polymers comprise a plurality of polymer side chains linked to a linear polymer backbone, the polymer side chains each comprising a hydrophilic polymer segment that has first and second ends, the first end of the hydrophilic polymer segment covalently linked to the linear polymer backbone, a cyclic anhydride residue having first and second ends, the first end of the cyclic anhydride residue covalently linked to the second end of the hydrophilic polymer segment, and a reactive moiety that is covalently linked to the second end of the cyclic anhydride residue.

Reactive bottlebrush polymers in accordance with the present disclosure include polymers having from 3 to 100 side chains, for example ranging anywhere from 3 to 4 to 5 to 6 to 7 to 8 to 10 to 12 to 15 to 20 to 25 to 50 to 75 to 100 side chains (in other words, having a number of side chains ranging between any two of the preceding values).

Hydrophilic polymer segments for the side chains can be selected from any of a variety of synthetic, natural, or hybrid synthetic-natural hydrophilic polymer segments. Examples of hydrophilic polymer segments include those that are formed from one or more hydrophilic monomers selected from alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars. Iodine- or bromine-substituted counterparts of the preceding monomers may be employed in some embodiments, to provide the final polymer with radiopacity.

Hydrophilic polymer segments may be selected, for example, from the following polymer segments: polyether segments including poly(alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(dimethyl acrylamide) segments, poly(N-vinylimidazole) segments, poly(4-vinylimidazole) segments, and poly(sodium 4-vinylbenzenesulfonate) segments, among others, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, protein segments, or polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties. Polymer segments for use in the bottlebrush and multi-arm polymers of the present disclosure typically contain between 10 and 1000 monomer units or more. Iodine- or bromine-substituted counterparts of the preceding polymer segments may be employed in some embodiments to provide the final polymer with radiopacity.

As previously noted, in the reactive bottlebrush polymers of the present disclosure, side chains that comprise hydrophilic polymer segments extend from a polymer backbone, typically a linear polymer backbone.

In some embodiments, side chain precursor molecules in the form of macromonomers (or macromers) that comprise a hydrophilic polymer segment and a terminal polymerizable group are first formed. Subsequently, the terminal polymerizable groups of the macromonomers are polymerized, optionally, in the present of one or more additional monomers, to form a precursor bottlebrush polymer having a linear polymer backbone and side chains comprising hydrophilic polymer segments extending from the linear polymer backbone.

In some embodiments, the side chains that comprise hydrophilic polymer segments are polymerized from initiator sites along a length of a linear precursor polymer backbone, thereby forming a precursor bottlebrush polymer having a linear polymer backbone and side chains comprising hydrophilic polymer segments extending from the linear polymer backbone.

The ends of at least a portion of the side chains of the precursor bottlebrush polymers are provided with reactive moieties. In some embodiments, the reactive moieties comprise electrophiles. Electrophiles may be selected, for example, from cyclic imide ester groups, such as succinimide ester groups, maleimide ester groups, glutarimide ester groups, phthalimide ester groups, bicyclo[2.2.1]hept-5-ene-2,3-dicarboxylic acid imide ester groups, imidazole ester groups, imidazole carboxylate groups and benzotriazole ester groups, among other possibilities. In other embodiments, the reactive moiety comprises an alkenyl-containing group, such as a vinyl group, an acryloyl group or a methacryloyl group.

In some embodiments, a hydrolysable ester group is provided between the reactive moiety and the hydrophilic polymer segment.

For example, the side chains of the bottlebrush polymers may comprise a hydrophilic polymer segment linked to the polymer backbone, a cyclic anhydride residue that is covalently linked to the hydrophilic polymer segment, and a reactive moiety that is covalently linked to the cyclic anhydride residue. In more particular embodiments, the side chains of the bottlebrush polymers may comprise a hydrophilic polymer segment that has first and second ends, the first end of the hydrophilic polymer segment covalently linked to the polymer backbone, an cyclic anhydride residue having first and second ends, the first end of the cyclic anhydride residue covalently linked to the second end of the hydrophilic polymer segment, and a reactive moiety that is covalently linked to the second end of the cyclic anhydride residue.

Examples of cyclic anhydride residues include residues of glutaric anhydride, residues of succinic anhydride, residues of malonic anhydride, residues of adipic anhydride, and residues of diglycolic anhydride, among others.

In some embodiments, the cyclic anhydride residues are residues of iodine-containing cyclic anhydrides. Examples of iodine-containing cyclic anhydrides include cyclic anhydrides in which at least one ring carbon of the cyclic anhydride compound is substituted with iodine alone or an iodinated moiety. Examples include cyclic anhydride compounds in which at least one ring carbon is substituted with an iodinated moiety that comprises an iodinated aromatic group. Examples of iodinated aromatic groups include iodine-substituted monocyclic aromatic groups and iodine-substituted multicyclic aromatic groups, such as iodinated phenyl groups, iodinated naphthyl groups, iodinated anthracenyl groups, iodinated phenanthrenyl groups, or iodinated tetracenyl groups. The iodinated aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.). The iodinated aromatic groups may be directly linked to the ring carbon or may be linked to the ring carbon through any suitable linking moiety, which may be selected, for example, from an alkyl group, ether group, ester group, amide group, amine group, or carbonate group, among others.

Specific examples of iodinated aromatic groups include those that comprise one or more monocyclic or multicyclic aromatic structures, substituted with (a) one or more iodine groups (e.g., one two, three, four, five, six or more iodine atoms) and (b) optionally, one or more hydroxyl-containing groups independently selected from one or more hydroxyl groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from alkyl groups, ether groups, ester groups, amide groups, amine groups, or carbonate groups, among others.

A few specific examples of iodine-containing cyclic anhydrides for use in the present disclosure include the following iodine-containing glutaric anhydride compounds, among others: 4-(2,3,5-triiodophenyl)tetrahydropyran-2,6-dione, CAS #2357909-35-2,

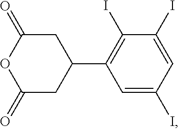 [SMILES: O=C1CC(c2cc(I)cc(I)c2I)CC(=O)O1]

4-(2-iodophenyl)tetrahydropyran-2,6-dione, CAS #2354202-92-7

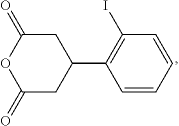 [SMILES: O=C1CC(c2ccccc2I)CC(=O)O1]

4-(3-iodophenyl)tetrahydropyran-2,6-dione, CAS #2353621-23-3,

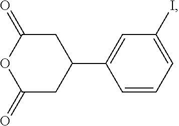 [SMILES: O=C1CC(c2cccc(I)c2)CC(=O)O1]

4-(4-iodophenyl)tetrahydropyran-2,6-dione, CAS #2354237-72-0,

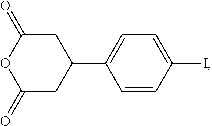 [SMILES: O=C1CC(c2ccc(I)cc2)CC(=O)O1]

4-((4-iodophenyl)methyl), tetrahydropyran-2,6-dione, CAS #2354625-91-3,

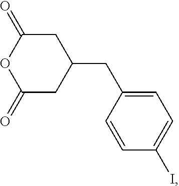 [SMILES: O=C1CC(Cc2ccc(I)cc2)CC(=O)O1]

4-1 (2-iodophenyl)methyl, tetrahydropyran-2,6-dione, CAS #2354597-51-4,

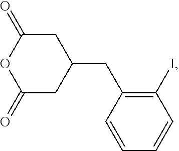 [SMILES: O=C1CC(Cc2ccccc2I)CC(=O)O1]

3-(2-iodophenyl)tetrahydrofuran-2,5-dione, CAS #887131-98-8,

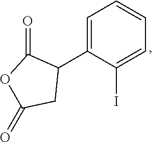 [SMILES: O=C1CC(c2ccccc2I)C(=O)O1]

3-(3-iodophenyl)tetrahydrofuran-2,5-dione, CAS #2353486-41-4,

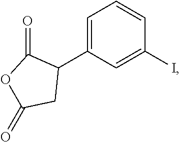 [SMILES: O=C1CC(c2cccc(I)c2)C(=O)O1]

3-(4-iodophenyl)tetrahydrofuran-2,5-dione, CAS #2354046-55-0,

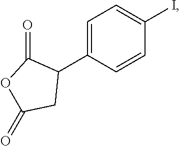 [SMILES: O=C1CC(c2ccc(I)cc2)C(=O)O1]

3-1(2-iodophenyl)methyltetrahydrofuran-2,5-dione, CAS #2353710-09-3,

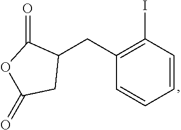 [SMILES: O=C1CC(Cc2ccccc2I)C(=O)O1]

3-((4-iodophenyl)methyl)tetrahydrofuran-2,5-dione, CAS #2352978-66-4

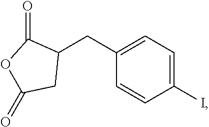 [SMILES: O=C1CC(Cc2ccc(I)cc2)C(=O)O1]

among others.

The above described non-iodinated and iodinated cyclic anhydrides, among others, may be reacted with a precursor bottlebrush polymer that comprises a linear polymer backbone and side chains comprising hydroxy-terminated hydrophilic polymer segments extending from the linear polymer backbone, under conditions of ring opening, to form a non-iodinated or iodinated carboxylic-acid-terminated precursor bottlebrush polymer comprising side chains that comprise a carboxylic acid end group that is linked to a hydrophilic polymer segment through a hydrolysable ester group.

A reactive moiety may then be linked to the non-iodinated or iodinated carboxylic-acid-terminated precursor bottlebrush polymer.

For example, an electrophilic moiety, such as a cyclic imide ester group, an imidazole ester group, an imidazole carboxylate group, or a benzotriazole ester group, may be linked to the non-iodinated or iodinated carboxylic-acid-terminated precursor bottlebrush polymer.

In particular embodiments, an N-hydroxy cyclic imide compound (e.g., N-hydroxysuccinimide (NHS), N-hydroxymaleimide, N-hydroxyglutarimide, N-hydroxyphthalimide, N-hydroxybicyclo[2.2.1]hept-5-ene-2,3-dicarboxylic acid imide (HONB),

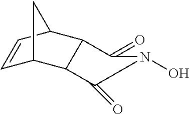 [SMILES: O=C1C2C3C=CC(C3)C2C(=O)N1O]

etc.) may be reacted with the non-iodinated or iodinated carboxylic-acid-terminated precursor bottlebrush polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a non-iodinated or iodinated reactive cyclic imide ester group (e.g., a non-iodinated or iodinated succinimide ester group, a non-iodinated or iodinated maleimide ester group, a non-iodinated or iodinated glutarimide ester group, a non-iodinated or iodinated phthalimide ester group, or a non-iodinated or iodinated bicyclo[2.2.1]hept-5-ene-2,3-dicarboxylic acid imide ester group, etc.) that is linked to a hydrophilic polymer segment through a hydrolysable ester group. In this way, a number of non-iodinated or iodinated reactive diester groups can be formed.

For example, in the particular case of N-hydroxysuccinimide as an N-hydroxy cyclic imide compound, exemplary reactive end groups include non-iodinated or iodinated succinimidyl malonate groups, non-iodinated or iodinated succinimidyl glutarate groups, non-iodinated or iodinated succinimidyl succinate groups, non-iodinated or iodinated succinimidyl adipate groups, and non-iodinated or iodinated succinimidyl diglycolate groups, among others. In the particular case of HONB as an N-hydroxy cyclic imide compound, exemplary reactive end groups include non-iodinated or iodinated bicyclo[2.2.1]hept-5-ene-2,3-dicarboxylic acid imidyl malonate groups, non-iodinated or iodinated bicyclo[2.2.1]hept-5-ene-2,3-dicarboxylic acid imidyl glutarate groups, non-iodinated or iodinated bicyclo[2.2.1]hept-5-ene-2,3-dicarboxylic acid imidyl succinate groups, non-iodinated or iodinated bicyclo[2.2.1]hept-5-ene-2,3-dicarboxylic acid imidyl adipate groups, and non-iodinated or iodinated bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include non-iodinated or iodinated maleimidyl malonate groups, non-iodinated or iodinated maleimidyl glutarate groups, non-iodinated or iodinated maleimidyl succinate groups, non-iodinated or iodinated maleimidyl adipate groups, and non-iodinated or iodinated maleimidyl diglycolate groups, among others. In the particular case of N-hydroxyglutarimide as an N-hydroxy cyclic imide compound, exemplary reactive end groups include non-iodinated or iodinated glutarimidyl malonate groups, non-iodinated or iodinated glutarimidyl glutarate groups, non-iodinated or iodinated glutarimidyl succinate groups, non-iodinated or iodinated glutarimidyl adipate groups, non-iodinated or iodinated glutarimidyl diglycolate groups, among others. In the particular case of N-hydroxyphthalimide as an N-hydroxy cyclic imide compound, exemplary reactive end groups include non-iodinated or iodinated phthalimidyl malonate groups, non-iodinated or iodinated phthalimidyl glutarate groups, non-iodinated or iodinated phthalimidyl succinate groups, non-iodinated or iodinated phthalimidyl adipate groups, and non-iodinated or iodinated phthalimidyl diglycolate groups, among others.

As another example, an alkenyl-containing group, such as a vinyl group, an acryloyl group or a methacryloyl group, may be linked to the non-iodinated or iodinated carboxylic-acid-terminated precursor bottlebrush polymer. Appending these functional groups can be accomplished through end group (amine or alcohol) reaction with either the acid chloride (e.g. methacryloyl chloride) or the acid anhydride (e.g. acrylic anhydride).

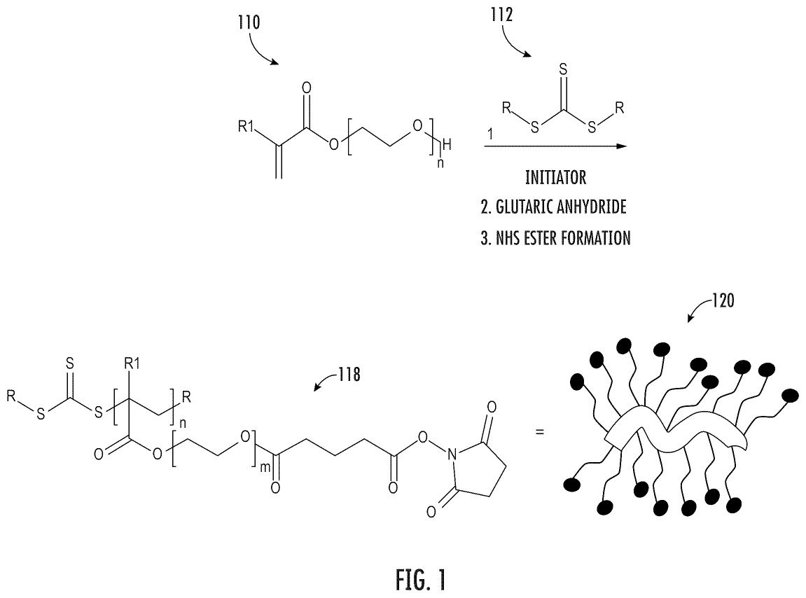

Various particular embodiments will now be described in conjunction with the drawings. FIG. 1 illustrates a method for forming a reactive bottlebrush polymer that comprises a plurality of side chains, which comprise polyalkylene oxide (specifically polyethylene oxide) segments, linked to a polyalkylene polymer backbone (specifically, a polyethylene backbone). Each side chain also comprises a reactive end moiety covalently linked to the polyalkylene oxide segment through a hydrolysable ester (specifically, a reactive end moiety comprising a succinimidyl ester group, more specifically, a succinimidyl glutarate group). In the embodiment shown, an acrylate or alkacrylate terminated PEG macromer is polymerized via reversible addition-fragmentation chain-transfer polymerization (RAFT polymerization) to form a reactive bottlebrush polymer precursor. RAFT polymerization is a type of living radical polymerization technique. RAFT makes use of a chain-transfer agent (RAFT agent) to mediate the polymerization via a reversible chain-transfer process. Examples of RAFT agents include, for example, dithioesters, xanthates, trithiocarbonates, dithiocarbamates, and dithiophosphonates.

In the specific examples shown in FIG. 1, an acrylate or C₁-C₄-alkacrylate terminated PEG macromer 110 (where R1 is-H or —C₁-C₄-alkyl and n ranges from 3 to 500) is subjected to RAFT polymerization in the presence of a trithiocarbonate RAFT initiator 112, where R is an alkyl, aryl, or cycloaliphatic functional group, to form a first precursor bottlebrush polymer having a linear acrylate or C₁-C₄-alkacrylate polymer backbone and side chains that hydrophilic polyethylene oxide segments extending from the linear polymer backbone, which polyethylene oxide segments comprise terminal hydroxyl groups. The hydroxy-terminated first precursor bottlebrush polymer is then reacted with glutaric anhydride via ring opening to produce a second precursor bottlebrush polymer having a linear acrylate or C₁-C₄-alkacrylate polymer backbone and side chains comprising glutaric-acid-end-capped polyethylene oxide segments extending from the linear polymer backbone. The glutaric-acid-end-capped second precursor bottlebrush polymer is then reacted with N-hydroxysuccinimide in the presence of a coupling agent to form a bottlebrush polymer 118 having a linear acrylate or C₁-C₄-alkacrylate polymer backbone and side chains comprising succinimidyl-glutarate-end-capped polyethylene oxide segments extending from the linear polymer backbone. In FIG. 1, m is an integer and may be a value ranging from 3 to 100.

Although an acrylate or C₁-C₄-alkacrylate terminated PEG macromer is illustrated in FIG. 1, acrylate or C₁-C₄-alkacrylate terminated macromers containing hydrophilic polymer segments other than PEG segments are envisioned.

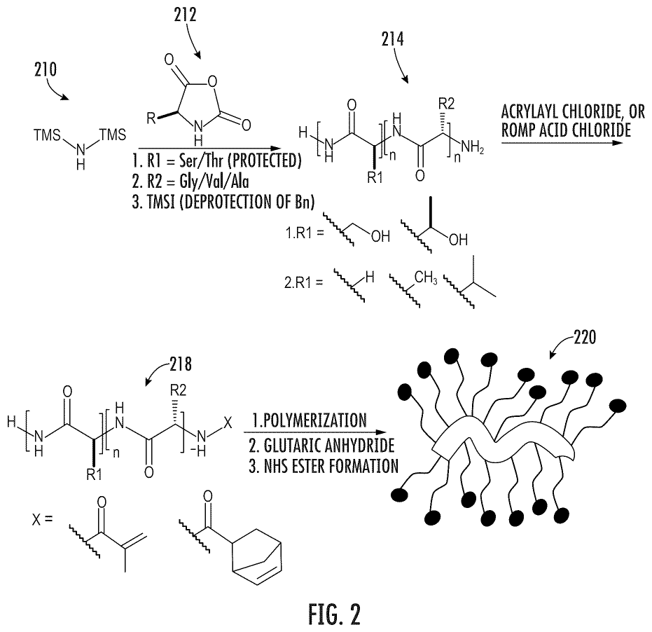

FIG. 2 describes a method for forming a reactive bottlebrush polymer that comprises a plurality of side chains comprising polyamide segments (specifically polyamino acid segments) linked to a polymer backbone (specifically, a polyacrylate, polymethacrylate or polynorbornene backbone). The side chains further comprise a reactive end moiety covalently linked to the side chain through a hydrolysable ester (specifically, an end moiety comprising a succinimidyl ester group, and more specifically, a succinimidyl glutarate group).

In the method of FIG. 2, a poly(amino acid) macromer is first formed followed by a suitable polymerization step. Poly(α-amino acid) macromers may be formed, for example, by first conducting a ring opening polymerization of a cyclic monomer such as an α-amino acid N-carboxyanhydride (NCA) monomer, which may be initiated by an amine, amine derivative, or organo-silicon compound, such as hexamethyldisilazane (HMDS), N-trimethylsilyl amine, or bis(trimethylsilyl)amine. In FIG. 2, bis(trimethylsilyl)amine 210 is used as initiator for synthesizing a block copolymer. In a first step, polymerization is conducted using an α-amino acid N-carboxyanhydride 212 such as L-serine N-carboxyanhydride (R=R1=hydroxymethyl) or L-threonine N-carboxyanhydride (R=R1=1-hydroxyethyl). This first polymerization step is followed by polymerization of another NCA such as L-glycine N-carboxyanhydride (R=R2=hydrogen), L-alanine N-carboxyanhydride (R=R2=methyl), or L-valine N-carboxyanhydride (R=R2=1-methylethyl).

This polymerization step is followed by deprotection of benzyl groups with trimethylsilyl iodide (TMSI), resulting in an amine-terminated copolymer 214 in which n is an integer and may independently range, for example, from 2 to 500, among other possibilities. The amine-terminated copolymer 214 is then provided with polymerizable end group(s), for example, by reaction with acryloyl chloride or methacryloyl chloride, or by reacting with a norbornene bound to an acid chloride and conducting ring-opening metathesis polymerization (ROMP) in the presence of an amine buffer to absorb the residual produced hydrochloric acid. The resulting macromer 218 is then polymerized, for example, by RAFT or ROMP, to form a first precursor bottlebrush polymer having a linear acrylate or methacrylate polymer backbone or a linear polynorbornene backbone and side chains that comprise poly(amino acid) segments extending from the linear polymer backbone, which poly(amino acid) segments comprise terminal amino groups. The amino-terminated first precursor bottlebrush polymer is then reacted with glutaric anhydride via an amide coupling reaction to produce a second precursor bottlebrush polymer having a linear polyacrylate, polymethacrylate or polynorbornene backbone and side chains comprising glutaric-acid-end-capped poly(amino acid) segments extending from the linear polymer backbone. The glutaric-acid-end-capped second precursor bottlebrush polymer is then reacted with N-hydroxysuccinimide in the presence of a coupling agent to form a bottlebrush polymer 220 having a linear polyacrylate, polymethacrylate or polynorbornene backbone and side chains comprising succinimidyl-glutarate-end-capped poly(amino acid) segments extending from the linear polymer backbone.

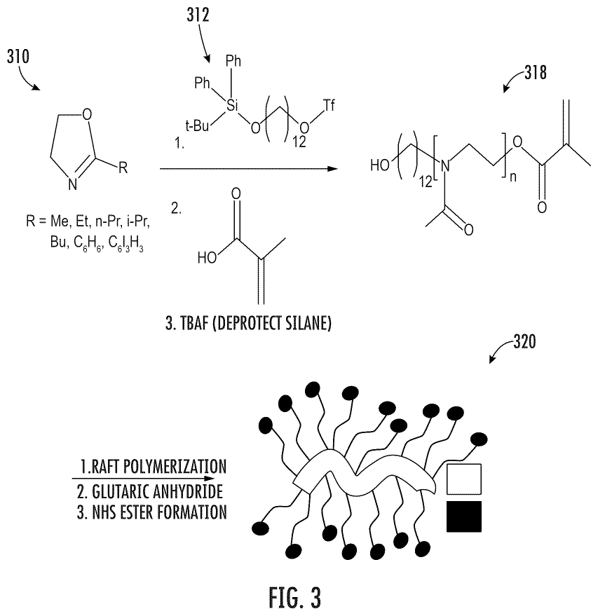

FIG. 3 describes a method for forming a reactive bottlebrush polymer that comprises a plurality of side chains comprising polyoxazoline segments linked to a polymer backbone (specifically, a polyacrylate, polymethacrylate or polynorbornene backbone) in which the side chains each have a reactive end moiety covalently linked to the side chain through a hydrolysable ester group (specifically, an end moiety comprising a succinimidyl ester group, more specifically, a succinimidyl glutarate group).

In the particular embodiment shown in FIG. 3, a polyoxazoline macromer is first formed followed by a suitable polymerization step. Polyoxazoline macromers may be formed, for example, by first polymerizing an oxazoline monomer 310, where R is selected from methyl, ethyl, n-propyl, i-propyl, phenyl, or triiodophenyl, among other possibilities, in the presence of a suitable initiator, for example, a p-toluenesulfonate compound such as methyl p-toluenesulfonate (MeOTs) or a trifluoromethanesulfonate compound such as methyl trifluoromethanesulfonate (MeOTf) or a tert-butyldiphenylsilyl protected 12-hydroxydodecyl trifluoromethanesulfonate compound 312. The resulting transient cationic terminated polymer is then capped and captured with a carboxylic acid. For example, the resulting polymer can be reacted with acrylic acid, methacrylic acid, or 5-norbornene carboxylic acid in a cationic capture to form an (meth)acrylate-terminated polymer or a norbornene-terminated macromonomer, followed by deprotection of the tert-butyldiphenylsilyl protected hydroxyl group by a fluoride source, such as tertbutyl amino fluoride [TBAF]. The resulting methacrylate-terminated macromonomer 318, acrylate-terminated macromonomer or norbornene-terminated macromonomer can then be polymerized, for example, in a RAFT polymerization process like that described above to form a first precursor bottlebrush polymer having a linear polyacrylate, polymethacrylate or polynorbornene backbone and side chains that comprise polyoxazoline segments extending from the linear polymer backbone, which polyoxazoline segments comprise terminal hydroxyl groups. The hydroxyl-terminated first precursor bottlebrush polymer is then reacted with glutaric anhydride to produce a second precursor bottlebrush polymer having a linear polyacrylate, polymethacrylate or polynorbornene backbone and side chains comprising glutaric-acid-end-capped polyoxazoline segments extending from the linear polymer backbone. The glutaric-acid-end-capped second precursor bottlebrush polymer is then reacted with N-hydroxysuccinimide in the presence of a coupling agent to form a bottlebrush polymer 320 having a linear polyacrylate, polymethacrylate or polynorbornene backbone and side chains comprising succinimidyl-glutarate-end-capped polyoxazoline segments extending from the linear polymer backbone. In FIG. 3, n is an integer and may range, for example, from 2 to 100, among other values.

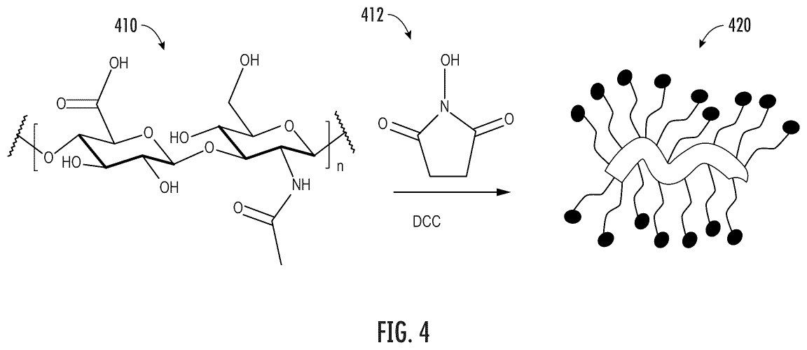

In another embodiment of the present disclosure shown in FIG. 4, a reactive polysaccharide, more particularly, a reactive hyaluronic acid polymer is formed. As shown in FIG. 4, carboxyl groups of a polysaccharide that contain one or more uronic acid species, more specifically, a hyaluronic acid polymer 410, are reacted with N-hydroxysuccinimide 412 in the presence of an ester coupling agent such as N,N′-dicyclohexylcarbodiimide (DCC), to form a polymer 420 having a hyaluronic acid backbone and reactive succinimidyl ester side groups. In some embodiments, only subset of —COOH groups are transformed into succinimidyl esters.

Reactive polymers in accordance with the present disclosure further include multi-arm and bottlebrush RAFT-derived hydrophilic polymers. These polymers comprise a multi-functional RAFT initiator residue and a plurality of polymer arms extending from the multi-functional RAFT initiator residue. The RAFT initiator may be selected from RAFT initiators having multiple functional groups (e.g., 3, 4, 5, 6, 7, 8, 9, 10, 12, 14, 16, 18, 20 or more functional groups), with the number of functional groups being selected based on the number of arms/side chains that are desired. For example, the RAFT initiator may contain multiple trithiocarbonate functional groups.

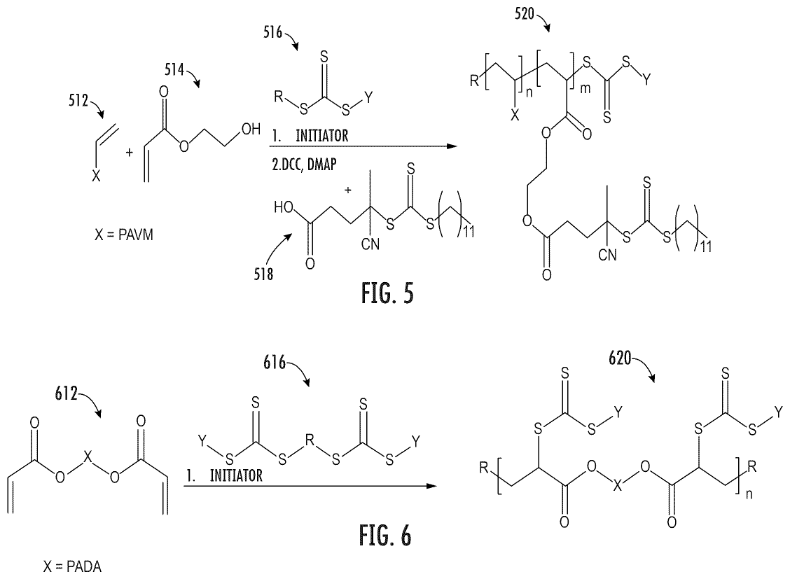

In a particular embodiment shown in FIG. 5, a polar aprotic vinyl monomer 512, where X is representing a range of polar aprotic vinyl monomers (PAVM), and an unsaturated hydroxylated monomer, specifically, 1-hydroxyethyl acrylate 514, are copolymerized in a RAFT polymerization in the presence of a trithiocarbonate initiator 516, where R=aliphatic, cycloaliphatic, or aromatic functional groups and Y=aliphatic, cycloaliphatic, or aromatic functional groups. The resulting copolymer is then reacted with 4-Cyano-4-[(dodecylsulfanylthiocarbonyl) sulfanyl]pentanoic acid 518 in the presence of a suitable coupling agent (e.g., a carbodiimide coupling agent such as N,N′-dicyclohexylcarbodiimide (DCC)) and a catalyst (e.g., 4-dimethylaminopyridine (DMAP)) to form a multi-functional RAFT agent 520, where n ranges from 5 to 5000 and m ranges from 2 to 100. RAFT agent 520, which is a linear polymer, can be used as an initiator in a subsequent RAFT polymerization process to provide a multi-arm/bottlebrush polymer.

In another particular embodiment shown in FIG. 6, a polar aprotic diacrylate monomer 612, where X is a radical representing a range of polar aprotic diacrylate monomers (PADA), is polymerized in a RAFT polymerization process in the presence of a trithiocarbonate initiator 616, where R=alkyl, aryl, or cycloaliphatic linker with or without polar functional groups promoting water solubility and Y=alkyl, aryl, cycloaliphatic to form a multi-functional RAFT agent 620, where n ranges from 1 to 100. RAFT agent 620, which is a linear polymer, can be used as an initiator in a subsequent RAFT polymerization process to provide a multi-arm/bottlebrush polymer in reactions with polar aprotic vinyl monomer (PAVM) feedstocks.

In further embodiments, a multi-functional RAFT agent, for example, a compound of the formula I, Bis-MPA-RAFT dendrimer (CAS: 2421217-33-4), may be used in a RAFT polymerization process to provide a multi-arm polymer.

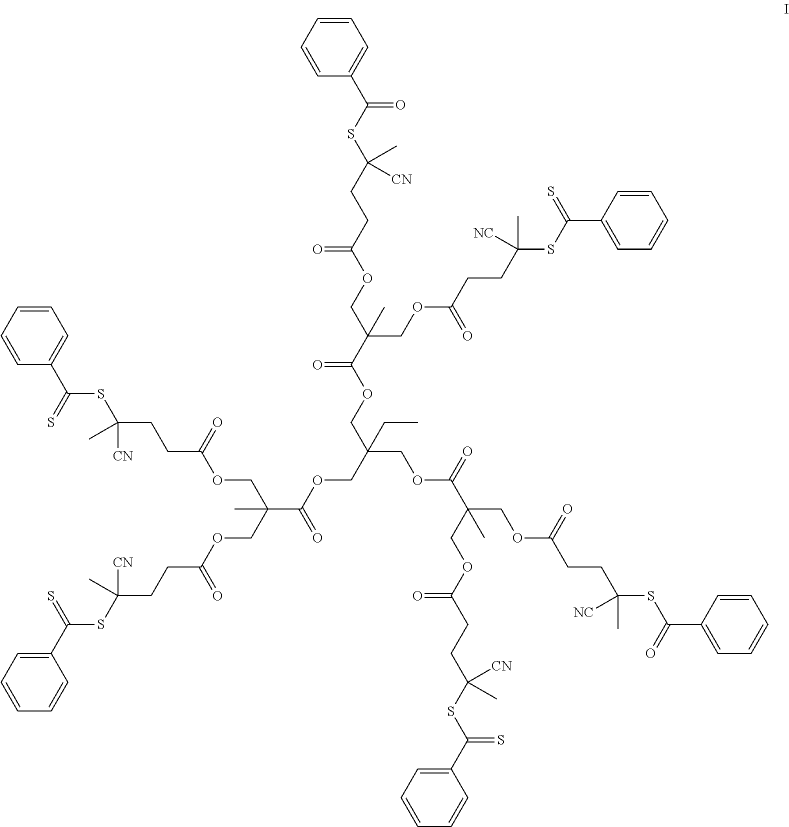 [SMILES: CCC(COC(=O)C(C)(COC(=O)CCC(C)(C#N)SC(=O)c1ccccc1)COC(=O)CCC(C)(C#N)SC(=S)c1ccccc1)(COC(=O)C(C)(COC(=O)CCC(C)(C#N)SC(=O)c1ccccc1)COC(=O)CCC(C)(C#N)SC(=S)c1ccccc1)COC(=O)C(C)(COC(=O)CCC(C)(C#N)SC(=S)c1ccccc1)COC(=O)CCC(C)(C#N)SC(=S)c1ccccc1]

Using the above and other RAFT initiators, RAFT polymerization may be conducted to form polymer segments from one or more polar aprotic vinyl monomers. Examples of polar aprotic vinyl monomers include, N-vinyl pyrrolidone, acrylamide, dimethyl acrylamide, N-vinylimidazole, 4-vinylimidazole, sodium 4-vinylbenzenesulfonate, among others.

Where the initiator comprises trithiocarbonate groups, the resulting polymer arms/side chains will contain terminal trithiocarbonate groups. In some embodiments, the trithiocarbonate-terminated polymer arms arms/side chains are subjected to aminolysis step, thereby forming thiol-terminated polymer arms/side chains, followed by a thiol-Michael addition reaction with an unsaturated hydroxylated compound such as hydroxyethyl acrylate, among other possibilities, thereby providing hydroxyl-terminated polymer arms/side chains. The hydroxyl-terminated polymer arms/side chains may then be reacted with glutaric anhydride, followed by N-hydroxysuccinimide to form a multi-arm polymer having a plurality of polymer arms/side chains comprising succinimidyl-glutarate-end-capped polymer segments, which segments are formed from one or more polar aprotic vinyl monomers, extending from a RAFT initiator residue.

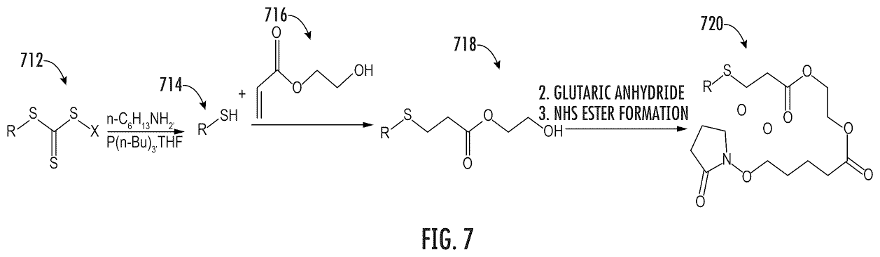

Aminolysis, thiol-Michael addition and succinimidyl glutarate end capping processes are illustrated in FIG. 7, in which a trithiocarbonate compound 712, where R is a previously described bottlebrush polymer formed from polar aprotic vinyl monomers and X is a sacrificial alkyl, aryl, or cycloaliphatic trithiocarbonate modifier is subjected to aminolysis using tributylphosphine (P (n-Bu) 3) and n-hexylamine (n-C₆H₁₃NH₂) in tetrahydrofuran (THF), thereby producing a thiol compound 714. Thiol compound 714 is then reacted with hydroxyethyl acrylate 716 in a Michael addition reaction to provide a compound 718 having a terminal hydroxyl group, which is then reacted with reacted with glutaric anhydride, followed by reaction with N-hydroxysuccinimide to provide a compound having a terminal succinimidyl glutarate group 720. Alternately, in place of N-hydroxysuccinimide, N-hydroxy-5-norbornene-2,3-dicarboxylic acid imide [HONB] may be used to form the electrophilic reactive group.

In various aspects, the present disclosure provides a hydrogel that comprises a crosslinked reaction product of (a) a reactive polymer as described hereinabove (e.g., a reactive bottlebrush polymer, a reactive multi-arm polymer, a reactive polysaccharide) and (b) a polyamine compound. For example, a reactive polymer comprising reactive cyclic imide ester groups may react with the amine groups of the polyamine compound under basic conditions, for example, at a pH ranging from about 7.4 to 11, more particularly, a pH ranging from about 9 to 11 in some embodiments to form an amide bond. As another example, a reactive polymer comprising reactive unsaturated groups (e.g., vinyl groups, acryloyl groups, methacryloyl groups, etc.) may react with the amino groups of the polyamino compound via Michael addition under basic conditions, for example, a pH ranging from about 7.4 to 11, more particularly, a pH ranging from about 9 to 11 in some embodiments to form an amine bond.

In general, polyamino compounds suitable for use in the present disclosure include, for example, small molecule polyamines (e.g., containing at least two amine groups, for instance, from 3 to 20 amine groups, or more, in certain embodiments), polymers having amine side groups, and branched polymers having amine end groups, including dendritic polymers having amine end groups. Polyamino compounds suitable for use in the present disclosure include those that comprises a plurality of —(CH₂)ₓ—NH₂ groups where x is 0, 1, 2, 3, 4, 5 or 6. Polyamino compounds suitable for use in the present disclosure include polyamino compounds that comprise basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Particular examples of polyamino compounds which may be used as the polyamino compound include ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, chitosan and derivatives thereof, and poly(allyl amine), among others among others.

In some embodiments, the polyamino compound may be substituted with one or more radiopaque atoms such as iodine or bromine. In particular embodiments, the polyamino compound may be substituted with iodine alone or an iodinated moiety, for example, selected from one of the iodinated moieties described above.

In various embodiments, the crosslinked reaction products of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including implants, medical devices, and pharmaceutical compositions.

In some aspects of the present disclosure, systems are provided that are configured to deliver (a) a polyamino compound and (b) a reactive polymer as described hereinabove (e.g., a reactive bottlebrush polymer, a reactive multi-arm polymer, or a reactive polysaccharide). The polyamino compound and the reactive polymer are combined under conditions such that the amino groups of the polyamino compound and the reactive moieties of the reactive polymer crosslink with one another. In certain embodiments, those conditions comprise an environment having a basic pH, for example, a pH ranging from about 7.4 to 11, more particularly, a pH ranging from about 9 to 11 in some embodiments. Such systems can be used to form crosslinked hydrogels, either in vivo or ex vivo.

In some aspects of the present disclosure, a system is provided that comprises (a) a first composition that comprises a polyamino compound, for example, as described herein and (b) a second composition that comprises a reactive polymer as described herein.

The first composition may be a first fluid composition comprising the polyamino compound or a first dry composition that comprises the polyamino compound, to which a suitable fluid such as water for injection, saline, etc. can be added to form a first fluid composition. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the polyamino compound is initially combined with the reactive polymer at an acidic pH at which crosslinking between the reactive moieties of the reactive polymer and the amino groups of the polyamino compound is suppressed. Then, when crosslinking is desired, a pH of the mixture of the polyamino compound and the reactive polymer is changed from an acidic pH to a basic pH, leading to crosslinking between same, thereby forming the crosslinked product.

In particular embodiments, the system comprises (a) a first composition that comprises a polyamino compound as described hereinabove, (b) a second composition that comprises a reactive polymer as described hereinabove, and (c) a third composition, specifically, an accelerant composition, that contains an accelerant that is configured to accelerate a crosslinking reaction between the polyamino compound and the reactive polymer.

The first composition may be a first fluid composition comprising the polyamino compound that is buffered to an acidic pH or a first dry composition that comprises the polyamino compound and acidic buffering composition, to which a suitable fluid such as water for injection, saline, etc. can be added to form a first fluid composition comprising the polyamino compound that is buffered to an acidic pH. In some embodiments, for example, the acidic buffering composition may comprise monobasic sodium phosphate, among other possibilities. The first fluid composition comprising the polyamino compound may have a pH ranging, for example, from about 3 to about 5. In addition to the polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the polyamino compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino compound that is buffered to an acidic pH and the second composition comprises a dry composition that comprises the reactive polymer. The first composition may then be mixed with the second composition to provide a prepared fluid composition that is buffered to an acidic pH and comprises the polyamino compound and the reactive polymer. In a particular example, a syringe may be provided that contains the first fluid composition comprising the polyamino compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that contains the polyamino compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (c) imageable radioisotopes including 99mTc, 201Th, 51Cr, 67Ga, 68Ga, 111 In, 64Cu, 89Zr, 59Fc, 42K, 82Rb, 24Na, 45Ti, 44Sc, 51Cr and 177Lu, among others, and (f) radiocontrast agents (beyond any radiopaque iodine or bromine atoms that are present) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

A prepared fluid composition that is buffered to an acidic pH and comprises the polyamino compound and the reactive polymer as described above, and a fluid accelerant composition that is buffered to basic pH as described above, may be combined form crosslinked hydrogels, either in vivo or ex vivo.

In various embodiments, a system is provided that includes one or more delivery devices for delivering first and second compositions to a subject.

In some embodiments, the system may include a delivery device that comprises a first reservoir that contains a first composition that comprises a polyamino compound as described above and a second reservoir that contains a second composition that comprises a reactive polymer that comprises a plurality of reactive moieties that are reactive with the amino moieties of the polyamino compound as described above.

In some embodiments, the system may include a delivery device that comprises a first reservoir that contains a first composition that comprises the polyamino compound and the reactive polymer and is buffered to an acidic pH, such as the prepared fluid composition previously described, and a second reservoir that contains second composition, such as the fluid accelerant composition previously described.

In either case, during operation, the first composition and second composition are dispensed from the first and second reservoirs and combined, whereupon the polyamino compound and the reactive polymer and crosslink with one another to form a crosslinked hydrogel.

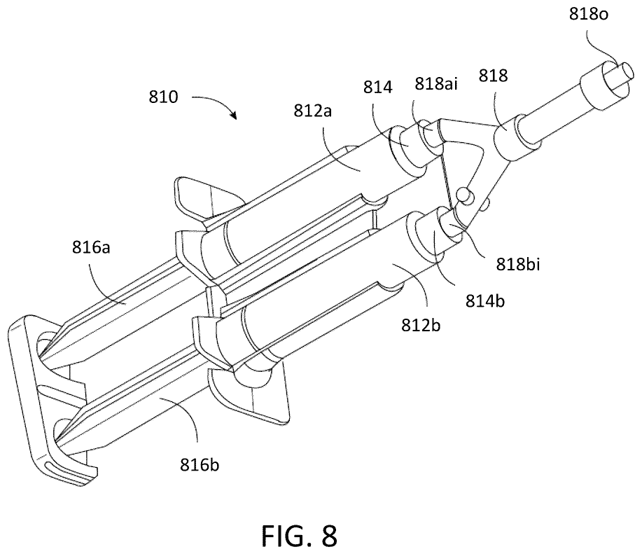

In particular embodiments, and with reference to FIG. 8, the system may include a delivery device 810 that comprises a double-barrel syringe, which includes first barrel 812a having a first barrel outlet 814a, which first barrel contains the first composition, a first plunger 816a that is movable in the first barrel 812a, a second barrel 812b having a second barrel outlet 814b, which second barrel 812b contains the second composition, and a second plunger 816b that is movable in the second barrel 812b. In some embodiments, the device 810 may further comprise a mixing section 818 having a first mixing section inlet 818ai in fluid communication with the first barrel outlet 814a, a second mixing section inlet 818bi in fluid communication with the second barrel outlet, and a mixing section outlet 8180.

In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. The imaging technique may be, for example, an ultrasonic imaging technique, a magnetic resonance imaging (MRI), or an x-ray-based imaging technique, such as computerized tomography or X-ray fluoroscopy.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel to a subject. Such systems may include one or more of the following: a syringe barrel, which may or may not contain crosslinked hydrogel particles as described herein; a vial, which may or may not contain crosslinked hydrogel particles as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel particles may be provided in dry form (e.g., powder form) or in a form that is ready for injection, such as an injectable hydrogel form (e.g., a suspension of crosslinked hydrogel particles).

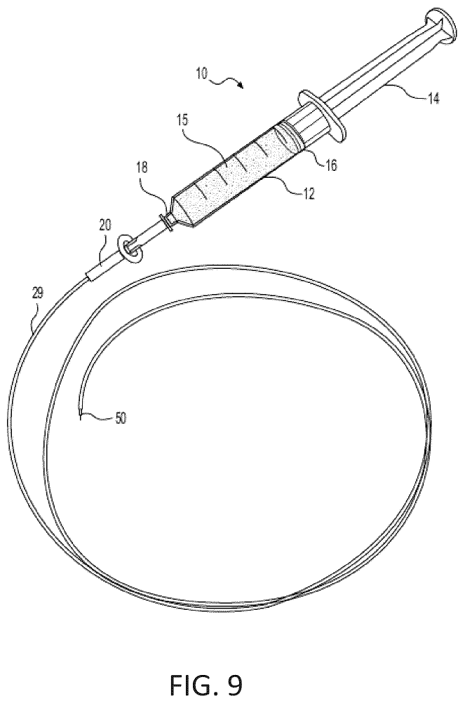

FIG. 9 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

After administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A reactive bottlebrush polymer comprising a polymer backbone and a plurality of polymer side chains linked to the polymer backbone, at least a portion of the side chains each comprising a hydrophilic polymer segment covalently linked to the polymer backbone and a reactive moiety covalently linked to the hydrophilic polymer segment at an end of the side chain opposite the polymer backbone.

2. The reactive bottlebrush polymer of claim 1, wherein the reactive moiety comprises an electrophilic group or alkenyl-containing group.

3. The reactive bottlebrush polymer of claim 1, wherein the reactive moiety comprises a cyclic imide ester group, an imidazole ester group, an imidazole carboxylate group, a benzotriazole ester group, an acryloyl group, or a methacryloyl group.

4. The reactive bottlebrush polymer of claim 1, wherein the hydrophilic polymer segment is selected from a polyethylene-oxide-containing segment, poly(amino acid)-containing segment, a polyoxazoline-containing segment, or a hydrophilic polymer segment containing one or more types of polar aprotic vinyl monomer residues.

5. The reactive bottlebrush polymer of claim 1, wherein the hydrophilic polymer segment is an iodine-containing hydrophilic polymer segment.

6. The reactive bottlebrush polymer of claim 1, further comprising a cyclic anhydride residue disposed between the hydrophilic polymer segment and the reactive moiety.

7. The reactive bottlebrush polymer of claim 6, wherein the cyclic anhydride residue is an iodine-containing cyclic anhydride residue.

8. The reactive bottlebrush polymer of claim 6, wherein the cyclic anhydride residue is selected from a residue of an iodine- or non-iodine-containing glutaric anhydride compound, a residue of an iodine- or non-iodine-containing succinic anhydride compound, a residue of an iodine- or non-iodine-containing malonic anhydride compound, a residue of an iodine- or non-iodine-containing adipic anhydride compound, and a residue of an iodine- or non-iodine-containing diglycolic anhydride compound.

9. A system for forming a hydrogel composition that comprises (a) polyamine compound and (b) a reactive bottlebrush polymer in accordance with claim 2.

10. The system of claim 9, wherein the system comprises a first composition that comprises the polyamine compound and a second composition that comprises the reactive bottlebrush polymer.

11. The system of claim 10, further comprising an accelerant composition.

12. The system of claim 9, further comprising a delivery device.

13. A crosslinked hydrogel composition comprising a crosslinked reaction product of (a) a polyamine compound and (b) a reactive bottlebrush polymer in accordance with claim 2.

14. A method of treatment comprising administering to a subject a mixture that comprises (a) a polyamine compound and (b) a reactive bottlebrush polymer in accordance with claim 2, under conditions such that the polyamine compound and the reactive bottlebrush polymer crosslink after administration.

15. A method of treatment comprising administering a reactive bottlebrush polymer in accordance with claim 2 to a body of a subject under conditions such that the reactive bottlebrush polymer reacts with amines found naturally in or on the body of the subject after administration.

16. A reactive multi-arm polymer comprising three or more polymer arms that each comprise a hydrophilic polymer segment containing one or more types of polar aprotic vinyl monomer residues and a reactive moiety covalently linked to the hydrophilic polymer segment.

17. The reactive multi-arm polymer of claim 16, wherein the reactive moiety comprises an electrophilic group or alkenyl-containing group.

18. The reactive multi-arm polymer of claim 16, wherein the reactive moiety comprises a cyclic imide ester group, an imidazole ester group, an imidazole carboxylate group, a benzotriazole ester group, an acryloyl group or a methacryloyl group.

19. A reactive polysaccharide comprising a polysaccharide backbone that comprises free carboxyl groups and cyclic imide ester groups covalently attached along a length of the polysaccharide backbone.

20. The reactive polysaccharide of claim 19, wherein the cyclic imide ester groups are selected from succinimide ester groups, maleimide ester groups, glutarimide ester groups, phthalimide ester groups, and bicyclo[2.2.1]hept-5-ene-2,3-dicarboxylic acid imide ester groups.
